(19) United States
(12) Patent Application Publication
(10) Pub. No.: US 2026/0144602 A1
(43) Pub. Date: May 28, 2026

(54) MEDICAL MICROSCOPY SYSTEM FOR IMAGING AN ANATOMICAL TARGET AREA OF A PATIENT

(71) Applicant: B. Braun New Ventures GmbH, Freiburg im Breisgau (DE)

(72) Inventor: Amir Sarvestani, Freiburg (DE)

(21) Appl. No.: 19/395,500

(22) Filed: Nov. 20, 2025

(30) Foreign Application Priority Data
Nov. 22, 2024 (DE) 10 2024 134 506.3

(51) Int. Cl. A61B 34/30; A61B 17/34; A61B 90/20; G06T 7/564

(52) U.S. Cl. CPC A61B 34/30; A61B 17/3423; A61B 90/20; G06T 7/564; A61B 2034/305; G06T 2207/10056

(57) ABSTRACT

A medical microscopy system for capturing an anatomical target area of a patient includes a cylindrical sleeve that can be arranged above the anatomical target area. The system also includes an imaging unit, an articulated arm robot unit, and an evaluation unit configured to determine a deviation of the contour of the front side from a target contour of a front side of the cylindrical sleeve in a captured image. The evaluation unit is also configured to determine a movement command for correcting a pose of the articulated arm robot unit based on the determined deviation. The articulated arm robot unit is configured to move at least one axis of the articulated arm robot unit based on the determined movement command.

### CROSS-REFERENCE TO RELATED APPLICATION

This application claims priority under 35 U.S.C. § 119 to German Application No. 10 2024 134 506.3, filed on Nov. 22, 2024, the content of which is incorporated by reference herein in its entirety.

### FIELD

The present disclosure relates to a medical microscopy system for imaging an anatomical target area of a patient in a medical operating room, and to the use of an articulated arm robot unit and/or an imaging unit in such a medical microscopy system.

### BACKGROUND

Microscopy systems are generally known from the prior art and are used in medical operating rooms, among other places, to image an anatomical target area or to image a patient or part of a patient, for example, exposed organs.

An access port can be used for an operation. The access port is usually placed on the patient over an open area of the body or on the body surface. The access port is usually implemented using a cylindrical sleeve. A surgeon then operates on an exposed organ, for example, through this access port. He looks through the access port. He can be assisted by a microscope that captures images of the area to be operated on (e.g., an organ) and its surroundings (e.g., tissue or arteries) through the access port and simultaneously displays the captured image on a screen.

DE 10 2015 103 426 A1 describes a microscope system and a method for automatically aligning a microscope.

In this context, it has now become apparent that there is a further need to provide a medical microscopy system for imaging an anatomical target area of a patient in a medical operating room.

### SUMMARY

It is therefore the purpose of the present disclosure to provide a medical microscopy system for imaging an anatomical target area of a patient in a medical operating room. In particular, the present disclosure is intended to provide an efficient medical microscopy system for imaging an anatomical target area in medical operating rooms, which enables precise alignment of the medical microscopy system with respect to the object.

A first aspect of the present disclosure relates to a medical microscopy system for imaging an anatomical target area of a patient in a medical operating room, wherein a cylindrical sleeve is arranged above the anatomical target area or on a surface of the anatomical target area, with an imaging unit that is configured to capture an image of the anatomical target area through the cylindrical sleeve, wherein the image comprises at least one partial section of a contour of a front side of the cylindrical sleeve, an articulated arm robot unit, wherein the imaging unit is arranged on the articulated arm robot unit, and an evaluation unit, which is configured to determine in the captured image a deviation of the at least one partial section of the contour of the front side from a target contour of a front side of the cylindrical sleeve, wherein the evaluation unit is configured to determine, on the basis of the determined deviation, a movement command for correcting a pose or position and/or orientation of the articulated arm robot unit so that the deviation is reduced, and wherein the articulated arm robot unit is configured to move at least one axis of the articulated arm robot unit based on the determined movement command in order to enable an alignment of the imaging unit so that the deviation is reduced.

The term “anatomical target area” should be understood broadly in this context and refers to a part of a patient. The anatomical target area may be, for example, an organ, a bone, or tissue of a patient. The object is preferably located beneath a patient's skin surface, wherein the skin surface has already been opened.

In this context, the term “patient” refers primarily to a human being or an animal.

In this context, the term “imaging unit” refers to an imaging device that is capable of capturing a two-dimensional or three-dimensional image and making it available for further processing, for example for display on a screen. The imaging unit may comprise, for example, a stereo microscope. The imaging unit may preferably be configured to capture the image in real time. The image can, for example, be displayed on the screen in real time. Real time here means a delay in the millisecond range.

In this context, the term “medical operating room” refers specifically to an operating room.

The term “cylindrical sleeve for an access port” refers in particular to a hollow cylinder that is placed over an open area of the body during surgery, through which a surgeon can insert instruments (e.g., a scalpel) during an operation. Such operations are also referred to as minimally invasive surgeries. Such a sleeve can also be referred to as a trocar sleeve. Preferably, the medical microscopy system may comprise a cylindrical sleeve for an access port. Preferably, the cylindrical sleeve can be configured to be positioned over an anatomical target area.

The term “articulated arm robot unit” refers in particular to an articulated arm robot with several movable axes, a control system, and rotary encoders for capturing the position and/or orientation of the end effector. The end effector in this case is the imaging unit. This means that the imaging unit is located on a terminal robot flange of the articulated arm robot unit. The articulated arm robot can be operated in automated, semi-automated, or manual mode. The articulated arm robot unit can preferably have an interface for attaching the imaging unit as an end effector.

The term “evaluation unit” refers in particular to a computing unit or processor that is configured to determine a deviation between a contour in the captured image and a target contour and, based on the deviation, to determine a movement command for the articulated robot unit in order to reduce, in particular minimize, the deviation.

In this context, the term “target contour” refers in particular to a circle. When the imaging unit is aligned directly above the cylindrical sleeve, with the imaging unit and the cylindrical sleeve aligned concentrically with each other or coaxially/with a common longitudinal axis, the contour of the cylindrical sleeve appears as a perfect circle on the image. In other words, any deviation from this concentric alignment of the imaging unit and cylindrical sleeve results in a deviation of the contour from the target contour. The contour is then displayed in a distorted manner.

In this context, the term “movement command” refers in particular to one or more commands for controlling one or more movement axes of the articulated arm robot unit.

The disclosure is based on the realization that an imaging unit designed to capture an image through a cylindrical sleeve should ideally be aligned concentrically with it. This means that the axis of the cylindrical sleeve and the axis of the imaging unit in/with which the image is captured are thus aligned. Any deviation from this ideal case will result in image distortion. Furthermore, only part of the anatomical target area may be captured, as both axes are at too great an angle to each other or are too skewed, and the image depicts the inner surface of the cylindrical sleeve rather than the bottom surface of the sleeve and the anatomical target area below it.

The disclosed microscopy system solves this problem by advantageously analyzing the captured image and determining a deviation of the captured contour of the cylindrical sleeve from a target contour. This can be done, for example, using an image analysis method (e.g., segmentation, feature recognition). The position of the imaging unit can be determined based on the deviation detected. Based on this determined position, it is possible to calculate how the imaging unit must be moved in order to minimize the deviation. To this end, movement commands for the articulated arm robot unit are then determined so that the imaging unit is moved into a better position, which enables the deviation to be reduced. This enables a better overall picture that does not show any distortion. Furthermore, this can better support a surgeon in his work. Furthermore, this does not require tracking of the cylindrical sleeve in order to determine the orientation and position of the sleeve. Overall, this provides an efficient and simple way to enable precise alignment of the microscopy system.

In a preferred embodiment, the evaluation unit can determine the deviation using an image analysis method (or machine vision method or machine vision).

The term “image analysis method” refers in particular to automated methods that are suitable for capturing and interpreting visual information from images. The image analysis method may include one or more of the following: Image segmentation, feature extraction, object recognition and classification, pattern recognition. In this way, the deviation can be determined automatically, very accurately, and without additional aids or human intervention. This makes it possible to dispense with additional tracking of the sleeve, which is advantageous.

Preferably, the evaluation unit determines the deviation by means of image segmentation.

According to a preferred embodiment, the target contour of the front side of the cylindrical sleeve may correspond to an ideal circle.

The ideal circle can be simply described by a constant radius around a center point. It is not necessary to specify the exact radius at first, as the size of the circle changes with the distance to the imaging unit, even if this is already ideal. This simple information, namely a constant radius, can be used to define a target contour in the image for the cylindrical sleeve. The deviation can now be determined, for example, by determining the center point and evaluating the radius. In this way, for example, the smallest radius and the largest radius can be determined. Such an evaluation can be used, for example, to determine an axis around which the articulated arm robot unit must rotate the imaging unit in order to minimize the deviation. For example, a constant increment can be rotated in each case, so that minimization can already be achieved with this simple analysis. The minimization can be performed iteratively until the determined deviation is below a predefined limit value (e.g., 0.1% deviation from the ideal circle). This can enable efficient determination of a movement command to minimize deviation.

According to a preferred embodiment, the evaluation unit can determine a first axis and a second axis perpendicular to the first axis in the captured at least one partial section of the contour of the front side, wherein the first axis and the second axis pass through a center point of the contour of the front side, wherein the first axis is selected such that it has a maximum length within the contour of the front side, wherein the movement command comprises a rotation about the first axis. This means that a rotational axis is formed by the first axis.

The center point, the first axis, and the second axis can each be determined using an image analysis method or machine vision method. The first axis determined in this way provides information about a virtual axis around which the articulated arm robot must rotate the imaging unit in order to minimize the deviation. Based on this virtual axis, movement commands can be derived for the articulated arm robot, which rotate the imaging unit around this virtual axis. In other words, the first axis and the second axis correspond to the two main axes of the ellipse.

For this purpose, the distance between the imaging unit and the center point of the anatomical target area can be determined via the imaging unit. The imaging unit can be a stereo microscope that provides this information (i.e., depth information). Furthermore, the fact that a virtual line between the center point of the imaging unit (i.e., end effector) and the center point of the contour coincides with the second axis of the contour can be utilized here. Furthermore, for example, the first axis can now be rotated at a constant distance by a predetermined angle increment in a circular path around the center point in the direction of the second axis. About the two positions (center point contour, end effector position), axis of rotation (i.e., first axis). The direction of rotation (i.e., second axis) and predetermined angular increment can be used to determine the position of the end effector in space. This position is then translated into movement commands (i.e., rotational movements of the individual articulated arms) and made available to the movement axes. The angle increment can be fixed, for example, 0.1°, 1°, 2°, etc. Alternatively, an angle can be determined from the ratio of the lengths of the first axis and the second axis of the contour and the positions of the center point of the contour and the end effector, which must be rotated so that the imaging unit and the cylindrical sleeve are aligned with each other.

According to a preferred embodiment, the evaluation unit may be configured to determine an inner surface of the contour of the access port in the image and, based on the determined inner surface, to determine a direction for rotation about the first axis for the movement command. This means that a direction of rotation is formed by the first axis.

For example, the evaluation unit can identify the inner surface of a cylindrical sleeve using an image analysis method or machine vision method, such as segmentation. This information can be used, for example, to determine a direction of rotation for the rotation of the imaging unit around the first axis. For example, the direction of rotation for reducing or minimizing the deviation can be derived from the inner surface, in particular from size information about the inner surface distributed around the circumference of the contour. For example, the direction of rotation corresponds to the direction in which a larger inner surface can be identified.

According to a preferred embodiment, the evaluation unit may be configured to determine a deviation of the center point of the captured at least one partial section of the contour of the front side from a center point of the image; wherein the movement command comprises a movement such that the center point of the captured at least one partial section of the contour of the front side and the center point of the image coincide. In other words, the evaluation unit can be configured to determine/calculate a translational deviation of the contour of the front side or the sleeve in the image, wherein the movement command comprises a translational movement in the direction of an image center or such that the image is in the sleeve in the image center.

According to a preferred embodiment, the microscopy system may comprise a navigation unit (or a navigation system or a position determination unit) for capturing a position of the imaging unit and/or a position of a patient.

The navigation unit can, for example, comprise a camera unit for determining a pose or position and/or orientation of the imaging unit and/or a pose or position and/or orientation of the patient. The position can, for example, be determined advantageously without markers on the imaging unit and/or the patient using an image analysis method or machine vision method. Alternatively, the navigation unit can track a marker placed on the patient to determine their position. Alternatively, the navigation unit can track a marker located on the imaging unit to determine its position. With the help of the position of the imaging unit and/or the position of the patient, a model of the anatomical target area (e.g., CT image, MRI image), which is displayed alongside the real-time image, for example, can be aligned accordingly. This can have a positive effect on a surgical procedure if both the real-time imaging of the anatomical target area and a corresponding model of the anatomical target area are displayed side by side and, preferably, aligned with each other.

According to a preferred embodiment, the microscopy system may include a display unit that is configured to display the image.

The display unit can, for example, advantageously display the image. This can advantageously increase the efficiency of the surgical procedure. The display unit may include a screen.

According to a preferred embodiment, the display unit may be configured to display a model of the anatomical target area and the cylindrical sleeve, in particular with the image and the model aligned with each other.

The model may include a preoperative image, such as an MRI image or a CT image.

This provides efficient support for the surgeon during the procedure, as both a high-quality, real-time image and a model can be displayed in an aligned position.

According to a preferred embodiment, the medical microscopy system may comprise an input unit via which the alignment of the imaging unit can be started and/or performed continuously.

The input unit can be a switch, an HMI (human-machine interface), or a touch display. The input unit can be used to trigger the alignment process, so that it is only carried out when it is really necessary. This can be advantageous in terms of conserving resources.

According to a preferred embodiment, the evaluation unit can determine the deviation based on geometry data of the cylindrical sleeve.

The geometry data may include a radius, a height, and a wall thickness of the cylindrical sleeve. Preferably, the geometry data includes a radius. The geometry data can be stored, for example, in an internal memory of the microscopy system or in an external memory accessed by the microscopy system. The geometry data can be used to further improve the accuracy of the alignment.

According to a preferred embodiment, the imaging unit may be configured to automatically perform a zoom function during alignment so that the deviation can be better determined.

The zoom function can be used to enlarge or reduce the contour in the image so that a contour is completely visible and/or analyzable. The zoom function can be further optimized using geometry data from the cylindrical sleeve. Furthermore, a predetermined rule can be used to automate the zoom function, for example. For example, it can be predefined that a contour detected in the image should make up a specific proportion, for example 95%, of the image. This can increase the manual adjustment effort for a user while simultaneously improving the quality of the alignment and the corresponding image.

According to a preferred embodiment, the imaging unit may be configured to automatically perform a zoom function after alignment, so that the anatomical target area is captured in an enlarged view.

By enlarging the anatomical target area while simultaneously aligning the imaging, the surgeon can advantageously be displayed a high-quality image of the anatomical target area in real time.

According to a preferred embodiment, the cylindrical sleeve may have an (optical) pattern. The pattern is used in particular for tracking the sleeve by means of a (visual) camera using machine vision.

The pattern may, for example, be applied to the front side of the cylindrical sleeve, to an outer surface of the cylindrical sleeve, and/or to an inner surface of the cylindrical sleeve. In this case, the pattern refers to a pattern known to the image analysis method. In this way, the efficiency and accuracy of determining the deviation of at least one partial section of the contour of the front side from a target contour of the front side of the cylindrical sleeve can be increased.

According to a preferred embodiment, the evaluation unit may be configured to receive geometry data of the cylindrical sleeve and, based on the geometry data of the cylindrical sleeve in the captured image, to determine a deviation of at least one partial section of the contour of the front side from a target contour of a front side of the cylindrical sleeve.

The geometry data refers, for example, to the outer radius, the inner radius, and/or the length of the cylindrical sleeve. In this way, the efficiency and accuracy of determining the deviation of at least one partial section of the contour of the front side from a target contour of the front side of the cylindrical sleeve can be increased.

Another aspect of the present disclosure relates to the use of an articulated arm robot unit and/or an imaging unit in a medical microscopy system described in more detail above.

The units according to one or more exemplary embodiments may be implemented using hardware, software, and/or a combination thereof. The units can be single-piece or multi-piece. Hardware units can be implemented, for example, by processing circuits such as a processor, a central processing unit (CPU), a controller, an arithmetic logic unit (ALU), a digital signal processor, a microcomputer, a field-programmable gate array (FPGA), a system-on-chip (SoC), a programmable logic device, a microprocessor, or any other device capable of responding to commands and executing them in a specified manner.

The units may comprise one or more interface circuits. In some examples, the interface circuits may include wired or wireless interfaces connected to a local area network (LAN), the Internet, a wide area network (WAN), or combinations thereof. The functionality of a specific unit of the present disclosure may be distributed across multiple units connected via interface circuits.

The units according to one or more exemplary embodiments may also include one or more storage devices. The one or more storage devices may be physical or non-transitory computer-readable storage media, such as random access memory (RAM), read-only memory (ROM), a permanent mass storage device (e.g., a hard disk drive), a solid-state device (e.g., NAND flash), and/or any other data storage mechanism capable of storing and recording data. The one or more storage devices may be used to store computer programs, program code, instructions, or a combination thereof.

The explanations and advantages of individual embodiments described here also apply mutatis mutandis to the other embodiments. Various exemplary features of the embodiments may be combined according to the present disclosure wherever this is technically useful and feasible.

In this context, it has now become apparent that there is a further need to provide a medical microscopy system for imaging an anatomical target area of a patient in a medical operating room.

### BRIEF DESCRIPTION OF THE DRAWINGS

FIG. 1 is a representation of a microscopy system for imaging an anatomical target area of a patient in a medical operating room of the present disclosure;

FIG. 2 shows an image with the outline of a cylindrical sleeve drawn on it;

FIG. 3 shows another image of a drawn contour of a cylindrical sleeve;

FIG. 4 shows a schematic analysis of the contour of a cylindrical sleeve;

FIG. 5 shows another image with an inner surface of a contour of a cylindrical sleeve; and

FIG. 6 shows an aligned imaging unit compared to a non-aligned imaging unit.

### DETAILED DESCRIPTION

The following describes exemplary embodiments of the present disclosure on the basis of the associated figures.

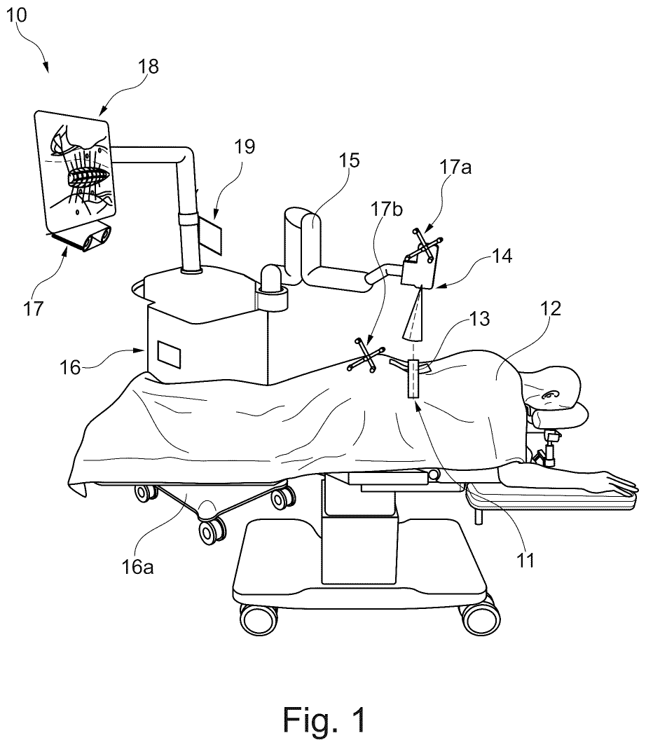

FIG. 1 shows a microscopy system 10 for imaging an anatomical target area 11 of a patient 12 in a medical operating room.

The microscopy system 10 has a cylindrical sleeve 13 for an access port. The cylindrical sleeve 13 is arranged above the anatomical target area 11. In particular, the cylindrical sleeve 13 may be configured to be positioned on or outside/above an object surface. The cylindrical sleeve 13 allows a surgeon to act on the anatomical target area, e.g., a tumor, using a medical instrument such as a scalpel.

The medical microscopy system 10 comprises an imaging unit 14. The imaging unit 14 is configured to capture an image of the anatomical target area 11 through the cylindrical sleeve 13. The imaging unit 14 is, in this case, a stereo microscope. The image comprises at least one partial section of a contour of a front side of the cylindrical sleeve 13.

The medical microscopy system 10 features an articulated arm robot unit 15. The imaging unit 14 is arranged on the articulated arm robot unit 15. The articulated arm robot unit 15 is arranged on a carrier vehicle 16a in the present case.

The medical microscopy system 10 further comprises an evaluation unit 16. The evaluation unit 16 is, in this case, a control unit located in the carrier vehicle 16a. The evaluation unit 16 is configured to determine, in the captured image, a deviation of at least one partial section of the contour of the front side from a target contour of a front side of the cylindrical sleeve 13. The target contour preferably corresponds to an (ideal) circle when the imaging unit 14 is ideally aligned above the cylindrical sleeve 13.

The evaluation unit 16 determines the deviation preferably by means of an image analysis method or machine vision method. To do this, it determines a first axis through a center point of the contour and a second axis perpendicular to the first axis. The first axis is determined so that it has a maximum length. In an ideal circle, both axes would be of equal length. However, if the imaging unit 14 is not arranged concentrically with a longitudinal axis of the cylindrical sleeve 13, the two axes are not of equal length. There is therefore a distortion. This distortion means that the lower part of the cylindrical sleeve 13 and thus the anatomical target area 11 are not optimally captured in the image.

The evaluation unit 16 is further configured to determine, based on the determined deviation, a movement command for correcting a position of the articulated arm robot unit so that the deviation is reduced. The evaluation unit 16 determines a position of the end effector in space based on the positions (center point of the contour, position of the end effector), axis of rotation (i.e., first axis), direction of rotation (i.e., second axis), and a predetermined angle increment. The evaluation unit 16 then derives movement commands (i.e., rotational movements of the individual articulated arms) for the articulated arm robot unit 15 from this position and makes these available to the articulated arm robot unit 15.

The articulated arm robot unit 15 then moves one or more axes of movement in accordance with the movement command in order to align the imaging unit 14 accordingly, so that the deviation is minimized. This process can be repeated iteratively until there is no longer any deviation or the deviation is smaller than a limit value.

Alternatively, the evaluation unit 16 can be configured to determine an angle from the ratio of the lengths of the first axis and the second axis of the contour and the positions of the center point of the contour and the end effector, around which the imaging unit must be rotated so that the imaging unit and the cylindrical sleeve 13 are aligned with each other.

The microscopy system 10 further comprises a navigation unit 17 for capturing a position of the imaging unit 14 and/or a position of a patient 12. For this purpose, markings 17a and 17b are applied to the imaging unit 14 and patient 12. With the help of the positions, for example, a model of the anatomical target area 11 can be aligned with the captured image of the anatomical target area on a display unit 18. The display unit 18 can, for example, display the captured image and the model side by side.

Furthermore, the microscopy system 10 comprises an input unit 19, via which the alignment of the imaging unit 14 can be started manually.

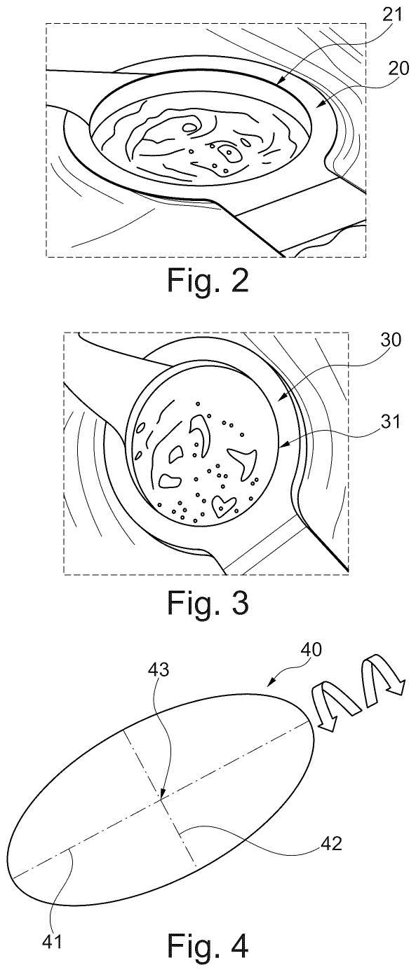

FIG. 2 shows an image captured by an imaging unit described in more detail above. The image shows the cylindrical sleeve 20 and a drawn contour 21 of the front side of the cylindrical sleeve 20. It is clearly visible that the contour 21 is distorted because the imaging unit 14 and the cylindrical sleeve 20 are not aligned with each other.

FIG. 3 shows an image captured by an imaging unit described in more detail above. The image shows the cylindrical sleeve 30 and a drawn contour 31 of the front side of the cylindrical sleeve 30. It is clearly evident that the contour 31 is not distorted, as the imaging unit 14 and the cylindrical sleeve 30 are aligned with each other.

FIG. 4 shows a schematic analysis of a contour 40 of a cylindrical sleeve. The first axis 41 and the second axis 42 perpendicular to it are plotted in each case. Both axes pass through the center point 43. The imaging unit must be rotated around the first axis 41, i.e., the longest axis, in the direction of the second axis 42 in order to reduce distortion.

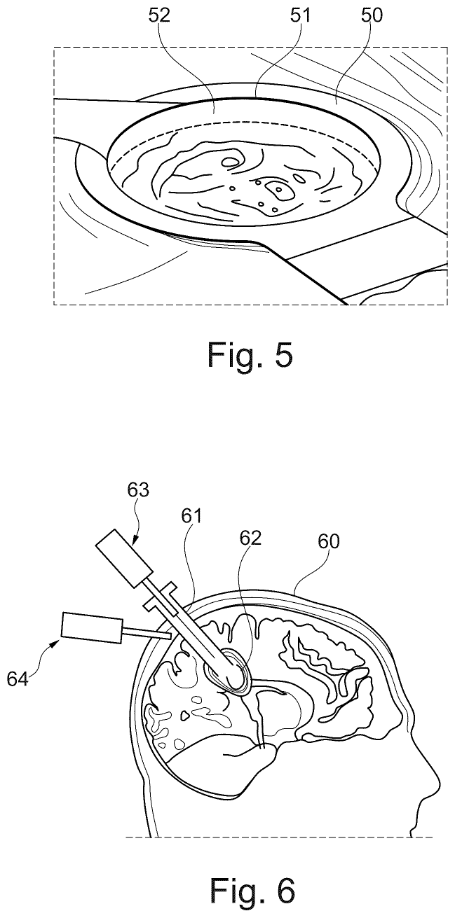

FIG. 5 shows an image with an inner contour 52 and contour 51 of the front side of a cylindrical sleeve 50 drawn in. The inner contour 52 provides information about the direction of rotation, in this case into the image plane, into which the imaging unit must be rotated in order to reduce deviation.

FIG. 6 shows an aligned imaging unit 63 compared to a non-aligned imaging unit 64. In this case, the anatomical target area 62 is a tumor in the head of a patient 60. The cylindrical sleeve 61 partially protrudes into the head of the patient 60. The imaging unit 64 is arranged at an angle to a longitudinal axis of the cylindrical sleeve 61. With the help of the microscopy system described in detail above, the misaligned imaging unit 64 could be brought into an aligned position.

### LIST OF REFERENCE SIGNS

10 Medical microscopy system
11, 62 Anatomical target area
12, 60 Patients
13, 20, 30, 50, 61 Cylindrical sleeve
14, 63, 64 Imaging unit
15 Articulated arm robot unit
16 Evaluation unit
21, 31, 40, 51 Contour
41 First axis
42 Second axis
43 Center point
52 Inner surface of the contour
17 Navigation unit
17a Imaging unit marking
17b Patient marking
18 Display unit
19 Input unit

1. A medical microscopy system for imaging an anatomical target area of a patient in a medical operating room, the medical microscopy system comprising:
a cylindrical sleeve configured for arrangement above the anatomical target area;
an articulated arm robot unit;
an imaging unit arranged on the articulated arm robot unit; and
an evaluation unit,
the imaging unit configured to capture an image of the anatomical target area through the cylindrical sleeve, with the image comprising at least one partial section of a contour of a front side of the cylindrical sleeve,
the evaluation unit configured to determine, in the image, a deviation of at least one partial section of the contour of the front side from a target contour of a front side of the cylindrical sleeve,
the evaluation unit further configured to determine, based on the deviation, a movement command for correcting a pose of the articulated arm robot unit so that the deviation is reduced, and
the articulated arm robot unit configured to move at least one axis of the articulated arm robot unit based on the movement command to enable alignment of the imaging unit with the cylindrical sleeve so that the deviation is reduced.

2. The medical microscopy system according to claim 1, wherein the evaluation unit determines the deviation by an image analysis method.

3. The medical microscopy system according to claim 1, wherein the target contour of the front side of the cylindrical sleeve corresponds to an ideal circle.

4. The medical microscopy system according to claim 1,
wherein the evaluation unit determines a first axis and a second axis perpendicular to the first axis in the at least one partial section of the contour of the front side,
wherein the first axis and the second axis pass through a center point of the contour of the front side,
wherein the first axis is selected so that the first axis has a maximum length within the contour of the front side, and
wherein the movement command comprises rotation about the first axis.

5. The medical microscopy system according to claim 4,
wherein the evaluation unit is configured to determine an inner surface of the contour of the cylindrical sleeve in the image and, based on the inner surface, to determine a direction for rotation about the first axis for the movement command.

6. The medical microscopy system according to claim 1,
wherein the evaluation unit is configured to determine a deviation of a center point of the at least one partial section of the contour of the front side from a center point of the image, and
wherein the movement command comprises a movement such that the center point of the at least one partial section of the contour of the front side coincides with the center point of the image.

7. The medical microscopy system according to claim 1, further comprising a navigation unit for capturing a position of the imaging unit and/or a position of a patient.

8. The medical microscopy system according to claim 1, further comprising a display unit configured to display the image.

9. The medical microscopy system according to claim 8, wherein the display unit is configured to display a model of the anatomical target area and the cylindrical sleeve.

10. The medical microscopy system according to claim 9, wherein the image and the model are aligned with each other.

11. The medical microscopy system according to claim 1, further comprising an input unit configured to align the imaging unit with the cylindrical sleeve.

12. The medical microscopy system according to claim 11, wherein the input unit is configured to start alignment of the imaging unit with the cylindrical sleeve and/or perform alignment continuously.

13. The medical microscopy system according to claim 1, wherein the evaluation unit is configured to determine the deviation based on geometry data of the cylindrical sleeve.

14. The medical microscopy system according to claim 1, wherein the imaging unit is configured to automatically perform a zoom function during alignment to assist determining the deviation.

15. The medical microscopy system according to claim 1, wherein the imaging unit is configured to automatically perform a zoom function after alignment so that the anatomical target area is captured in an enlarged view.

16. The medical microscopy system according to claim 1, wherein the cylindrical sleeve has a pattern.

17. The medical microscopy system according to claim 1, wherein the evaluation unit is configured to receive geometry data of the cylindrical sleeve and, based on the geometry data of the cylindrical sleeve in the image, to determine a deviation of at least one partial section of the contour of the front side from a target contour of a front side of the cylindrical sleeve.
